(19) United States
(12) Patent Application Publication
(10) Pub. No.: US 2021/0340561 A1
(43) Pub. Date: Nov. 4, 2021

(54) COMPOSITIONS AND METHODS FOR REPROGRAMMING SKIN INTO INSULIN PRODUCING TISSUE

(71) Applicant: Ohio State Innovation Foundation, Columbus, OH (US)

(72) Inventors: Daniel Gallego Perez, Columbus, OH (US); Chandan Sen, Columbus, OH (US); Natalia Higuita Castro, Columbus, OH (US)

(21) Appl. No.: 17/259,766

(22) PCT Filed: Aug. 1, 2019
(86) PCT No.: PCT/US2019/044718

§ 371 Date: Jan. 12, 2021

Related U.S. Application Data
(60) Provisional application No. 62/713,239, filed on Aug. 1, 2018.

(51) Int. Cl. C12N 15/85; C12N 5/071; C12N 15/87; A61P 3/10; A61K 35/36

(52) U.S. Cl. CPC C12N 15/85; C12N 5/0629; C12N 2510/00; A61P 3/10; A61K 35/36; C12N 15/87

(57) ABSTRACT

Disclosed herein are compositions and methods that involve using compositions for directly reprogramming skin cells into insulin-producing cells both in vitro and in vivo. These compositions and methods are useful for a variety of purposes, including the treatment of insulin-dependent and insulin-resistant diabetes. Therefore, also disclosed is a method for treating diabetes in a subject that involves reprogramming an effective amount of skin cells in the subject into an insulin producing cell using the method disclosed herein.

### CROSS-REFERENCE TO RELATED APPLICATIONS

This application claims benefit of U.S. Provisional Application No. 62/713,239, filed Aug. 1, 2018, which is hereby incorporated herein by reference in its entirety.

### SEQUENCE LISTING

This application contains a sequence listing filed in electronic form as an ASCII.txt file entitled “321501-2330 Sequence Listing_ST25” created on Aug. 1, 2019. The content of the sequence listing is incorporated herein in its entirety.

### BACKGROUND

Type 1 diabetes (T1D) is a chronic, debilitating autoimmune disease targeting pancreatic β-cells diminishing the β-cell mass resulting in insufficient insulin production. Although the exact etiology of T1D remains unknown, it is prevalent among young children and remains without a practical cure, despite being one of the most common endocrine disorders. Successful transplantation of pancreatic islets or the pancreas can reduce some complications of T1D. However, organ shortage severely limits this approach. Therefore, improved compositions and methods are needed to replenish endogenous insulin-producing cells.

### SUMMARY

Disclosed herein are compositions and methods for reprogramming skin cells into insulin-producing cells both in vitro and in vivo. One embodiment discloses a polynucleotide comprising two or more nucleic acid sequences encoding Pdx1, Ng3, Mafa, and Tcf3 (“PMN-T factors”). In some embodiments, the PMN-T factors are mammalian proteins, such as human proteins.

In some embodiments, the PMN-T factors are expressed at approximately equal ratios. In some embodiments, the Pdx1, Ng3, Mafa, and Tcf3 proteins are expressed at ratios of about 1:1:1:1, 2:1:1:1, 1:2:1:1, 1:1:2:1, 1:1:1:2, 2:2:1:1, 2:1:2:1, 2:1:1:2, 1:2:2:1, 1:1:2:2, 1:2:1:2, 3:1:1:1, 1:3:1:1, 1:1:3:1, 1:1:1:3, 3:2:1:1, 3:1:2:1, 3:1:1:2, 1:3:2:1, 1:1:3:2, 1:3:1:2, 2:3:1:1, 2:1:3:1, 2:1:1:3, 1:2:3:1, 1:1:2:3, 1:2:1:3 (Pdx1:Ng3:Mafa:Tcf3).

Also disclosed are non-viral vectors containing the disclosed polynucleotides. In particular embodiments, the vector is a recombinant bacterial plasmid. For example, in some embodiments, the non-viral vector has a pCDNA3 backbone. In some embodiments, the vector comprises an internal ribosome entry site (IRES).

Also disclosed is a method of reprogramming skin cells into insulin-producing cells that involves delivering intracellularly into the skin cells a polynucleotide comprising nucleic acid sequences encoding Pdx1, Ng3, Mafa, and Tcf3. Each of the PNM-T factors can be delivered simultaneously, sequentially, or any combination thereof. In some embodiments, the method involves first delivering intracellularly into the skin cells a polynucleotide comprising nucleic acid sequences encoding Pdx1. The method can then involve delivering intracellularly into the skin cells a polynucleotide comprising nucleic acid sequences encoding Ng3, Mafa, and Tcf3, 1, 2, 3, 4, 5, 6, 7, 8, 9, 10, 11, or 12, days later. In some embodiments, the method involves first delivering intracellularly into the skin cells a polynucleotide comprising nucleic acid sequences encoding Tcf3. The method can then involve delivering intracellularly into the skin cells a polynucleotide comprising nucleic acid sequences encoding Ng3, Mafa, and Pdx1, 1, 2, 3, 4, 5, 6, 7, 8, 9, 10, 11, or 12, days later. In some embodiments, the method involves first delivering intracellularly into the skin cells a polynucleotide comprising nucleic acid sequences encoding Mafa. The method can then involve delivering intracellularly into the skin cells a polynucleotide comprising nucleic acid sequences encoding Ng3, Tcf3, and Pdx1, 1, 2, 3, 4, 5, 6, 7, 8, 9, 10, 11, or 12, days later. In some embodiments, the method involves first delivering intracellularly into the skin cells a polynucleotide comprising nucleic acid sequences encoding Ng3. The method can then involve delivering intracellularly into the skin cells a polynucleotide comprising nucleic acid sequences encoding Mafa, Tcf3, and Pdx1, 1, 2, 3, 4, 5, 6, 7, 8, 9, 10, 11, or 12, days later.

In some embodiments, after transfecting target cells with nucleic acid sequences encoding the PMN-T factors, the cells can then pack the transfected genes (e.g. cDNA) into EVs, which can then assist in the formation of insulin-producing cells by other skin cells. Therefore, also disclosed is a method of reprogramming skin cells into insulin-producing cells that involves exposing the skin cells with an extracellular vesicle produced from a cell containing or expressing the PMN-T factors.

In these embodiments, the polynucleotides and compositions may be delivered to the skin cells, or the donor cells, intracellularly via a gene gun, a microparticle or nanoparticle suitable for such delivery, transfection by electroporation, three-dimensional nanochannel electroporation, a tissue nanotransfection device, a liposome suitable for such delivery, or a deep-topical tissue nanoelectroinjection device. In some of these embodiments, the polynucleotides can be incorporated into a non-viral vector, such as a bacterial plasmid. In some embodiments, a viral vector can be used. For example, the polynucleotides can be incorporated into a viral vector, such as an adenoviral vector. However, in other embodiments, the polynucleotides are not delivered virally.

Also disclosed is a method for treating diabetes in a subject that involves reprogramming an effective amount of skin cells in the subject into an insulin producing cell using the method disclosed herein. For example, in some embodiments, the subject has insulin-dependent diabetes. In some embodiments, the subject has insulin-resistant diabetes. In some embodiments, the subject has controlled blood sugar (is not hyperglycemic) during treatment. For example, in some embodiments, the subject has a fasting blood glucose level less than 180, 170, 160, 150, 140, 130, 120, or 110 mg/dL during treatment, including between 70 and 130 mg/dL during treatment.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1E are graphs showing blood glucose in mice with type 1 diabetes induced by streptozotocin injection after treatment with PBS (FIG. 1A), PNM factors (FIG. 1B), PNM-T factors (FIG. 10), Tcf3 alone (FIG. 1D), or Tcf3 at day 1 and PNM at day 7 (FIG. 1E) from week 1 until week 14.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

### Definitions

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “polypeptide” refers to amino acids joined to each other by peptide bonds or modified peptide bonds, e.g., peptide isosteres, etc. and may contain modified amino acids other than the 20 gene-encoded amino acids. The polypeptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given polypeptide. Also, a given polypeptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W. H. Freeman and Company, New York (1993); Posttranslational Covalent Modification of Proteins, B.C. Johnson, Ed., Academic Press, New York, pp. 1-12 (1983)).

As used herein, the term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof.

A “nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more nucleotides linked together. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but are linked together through a moiety other than a phosphate moiety. Nucleotide substitutes are able to conform to a double helix type structure when interacting with the appropriate target nucleic acid.

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

For purposes herein, the % sequence identity of a given nucleotides or amino acids sequence C to, with, or against a given nucleic acid sequence D (which can alternatively be phrased as a given sequence C that has or comprises a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software.

By “specifically hybridizes” is meant that a probe, primer, or oligonucleotide recognizes and physically interacts (that is, base-pairs) with a substantially complementary nucleic acid (for example, a c-met nucleic acid) under high stringency conditions, and does not substantially base pair with other nucleic acids.

The term “stringent hybridization conditions” as used herein mean that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C. Other hybridization and wash conditions are well known and are exemplified in Sambrook et al, Molecular Cloning: A Laboratory Manual, Second Edition, Cold Spring Harbor, N.Y. (1989), particularly chapter 11.

Disclosed herein are compositions and methods for reprogramming skin cells into insulin-producing cellsboth in vitro and in vivo.

Compositions

Disclosed are polynucleotides comprising nucleic acid sequences encoding proteins selected from the group consisting of Pdx1, Ng3, Mafa, and Tcf3 (“PMN-T factors”). The amino acid and nucleic acid sequences encoding Pdx1, Ng3, Mafa, and Tcf3 are known in the art.

In some embodiments, the Pdx1 comprises the amino acid sequence:

(SEQ ID NO: 1)
MNGEEQYYAATQLYKDPCAFQRGPAPEFSASPPACLYMGRQPPPPPPHP
FPGALGALEQGSPPDISPYEVPPLADDPAVAHLHHHLPAQLALPHPPAG
PFPEGAEPGVLEEPNRVQLPFPWMKSTKAHAWKGQWAGGAYAAEPEENK
RTRTAYTRAQLLELEKEFLFNKYISRPRRVELAVMLNLTERHIKIWFQN
RRMKVVKKEEDKKRGGGTAVGGGGVAEPEQDCAVTSGEELLALPPPPPP
GGAVPPAAPVAAREGRLPPGLSASPQPSSVAPRRPQEPR,

or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:1.

In some embodiments, the nucleic acid sequence encoding the Pdx1 comprises the nucleic acid sequence:

(SEQ ID NO: 2)
ATGAACGGCGAGGAGCAGTACTACGCGGCCACGCAGCTTTACAAGGACC
CATGCGCGTTCCAGCGAGGCCCGGCGCCGGAGTTCAGCGCCAGCCCCCC
TGCGTGCCTGTACATGGGCCGCCAGCCCCCGCCGCCGCCGCCGCACCCG
TTCCCTGGCGCCCTGGGCGCGCTGGAGCAGGGCAGCCCCCCTGACATCT
CCCCGTACGAGGTGCCCCCCCTCGCCGACGACCCCGCGGTGGCGCACCT
TCACCACCACCTCCCGGCTCAGCTCGCGCTCCCCCACCCGCCCGCCGGG
CCCTTCCCGGAGGGAGCCGAACCGGGCGTCCTGGAGGAGCCCAACCGCG
TCCAGCTGCCTTTCCCATGGATGAAGTCTACCAAAGCTCACGCGTGGAA
AGGCCAGTGGGCAGGCGGCGCCTACGCTGCGGAGCCGGAGGAGAACAAG
CGGACGCGCACGGCCTACACGCGCGCACAGCTGCTAGAGCTGGAGAAGG
AGTTCCTATTCAACAAGTACATCTCACGGCCGCGCCGGGTGGAGCTGGC
TGTCATGTTGAACTTGACCGAGAGACACATCAAGATCTGGTTCCAAAAC
CGCCGCATGAAGTGGAAAAAGGAGGAGGACAAGAAGCGCGGCGGCGGGA
CAGCTGTCGGGGGTGGCGGGGTCGCGGAGCCTGAGCAGGACTGCGCCGT
GACCTCCGGCGAGGAGCTTCTGGCGCTGCCGCCGCCGCCGCCCCCCGGA
GGTGCTGTGCCGCCCGCTGCCCCCGTTGCCGCCCGAGAGGGCCGCCTGC
CGCCTGGCCTTAGCGCGTCGCCACAGCCCTCCAGCGTCGCGCCTCGGCG
GCCGCAGGAACCACGA,

or a nucleic acid sequence that hybridizes to a nucleic acid sequence consisting of SEQ ID NO:2 under stringent hybridization conditions.

In some embodiments, the Ng3 comprises the amino acid sequence:

(SEQ ID NO: 3)
MTPQPSGAPTVQVTRETERSFPRASEDEVTCPTSAPPSPTRTRGNCAEA
EEGGCRGAPRKLRARRGGRSRPKSELALSKQRRSRRKKANDRERNRMHN
LNSALDALRGVLPTFPDDAKLTKIETLRFAHNYIWALTQTLRIADHSLY
ALEPPAPHCGELGSPGGSPGDWGSLYSPVSQAGSLSPAASLEERPGLLG
ATSSACLSPGSLAFSDFL,

or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:3.

In some embodiments, the nucleic acid sequence encoding the Ng3 comprises the nucleic acid sequence:

(SEQ ID NO: 4)
ATGACGCCTCAACCCTCGGGTGCGCCCACTGTCCAAGTGACCCGTGAGA
CGGAGCGGTCCTTCCCCAGAGCCTCGGAAGACGAAGTGACCTGCCCCAC
GTCCGCCCCGCCCAGCCCCACTCGCACACGGGGGAACTGCGCAGAGGCG
GAAGAGGGAGGCTGCCGAGGGGCCCCGAGGAAGCTCCGGGCACGGCGCG
GGGGACGCAGCCGGCCTAAGAGCGAGTTGGCACTGAGCAAGCAGCGACG
GAGTCGGCGAAAGAAGGCCAACGACCGCGAGCGCAATCGAATGCACAAC
CTCAACTCGGCACTGGACGCCCTGCGCGGTGTCCTGCCCACCTTCCCAG
ACGACGCGAAGCTCACCAAGATCGAGACGCTGCGCTTCGCCCACAACTA
CATCTGGGCGCTGACTCAAACGCTGCGCATAGCGGACCACAGCTTGTAC
GCGCTGGAGCCGCCGGCGCCGCACTGCGGGGAGCTGGGCAGCCCAGGCG
GTTCCCCCGGGGACTGGGGGTCCCTCTACTCCCCAGTCTCCCAGGCTGG
CAGCCTGAGTCCCGCCGCGTCGCTGGAGGAGCGACCCGGGCTGCTGGGG
GCCACCTCTTCCGCCTGCTTGAGCCCAGGCAGTCTGGCTTTCTCAGATT
TTCTG,

or a nucleic acid sequence that hybridizes to a nucleic acid sequence consisting of SEQ ID NO:4 under stringent hybridization conditions.

In some embodiments, the Mafa comprises the amino acid sequence:

(SEQ ID NO: 5)
MAAELAMGAELPSSPLAIEYVNDFDLMKFEVKKEPPEAERFCHRLPPGS
LSSTPLSTPCSSVPSSPSFCAPSPGTGGGGGAGGGGGSSQAGGAPGPPS
GGPGAVGGTSGKPALEDLYWMSGYQHHLNPEALNLTPEDAVEALIGSGH
HGAHHGAHHPAAAAAYEAFRGPGFAGGGGADDMGAGHHHGAHHAAHHHH
AAHHHHHHHHHHGGAGHGGGAGHHVRLEERFSDDQLVSMSVRELNRQLR
GFSKEEVIRLKQKRRTLKNRGYAQSCRFKRVQQRHILESEKCQLQSQVE
QLKLEVGRLAKERDLYKEKYEKLAGRGGPGSAGGAGFPREPSPPQAGPG
GAKGTADFFL,

or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:5.

In some embodiments, the nucleic acid sequence encoding the Mafa comprises the nucleic acid sequence:

(SEQ ID NO: 6)
ATGGCCGCGGAGCTGGCGATGGGCGCCGAGCTGCCCAGCAGCCCGCTGG
CCATCGAGTACGTCAACGACTTCGACCTGATGAAGTTCGAGGTGAAGAA
GGAGCCTCCCGAGGCCGAGCGCTTCTGCCACCGCCTGCCGCCAGGCTCG
CTGTCCTCGACGCCGCTCAGCACGCCCTGCTCCTCCGTGCCCTCCTCGC
CCAGCTTCTGCGCGCCCAGCCCGGGCACCGGCGGCGGCGGCGGCGCGGG
GGGCGGCGGCGGCTCGTCTCAGGCCGGGGGCGCCCCCGGGCCGCCGAGC
GGGGGCCCCGGCGCCGTCGGGGGCACCTCGGGGAAGCCGGCGCTGGAGG
ATCTGTACTGGATGAGCGGCTACCAGCATCACCTCAACCCCGAGGCGCT
CAACCTGACGCCCGAGGACGCGGTGGAGGCGCTCATCGGCAGCGGCCAC
CACGGCGCGCACCACGGCGCGCACCACCCGGCGGCCGCCGCAGCCTACG
AGGCTTTCCGCGGCCCGGGCTTCGCGGGCGGCGGCGGAGCGGACGACAT
GGGCGCCGGCCACCACCACGGCGCGCACCACGCCGCCCACCACCACCAC
GCCGCCCACCACCACCACCACCACCACCACCACCATGGCGGCGCGGGAC
ACGGCGGTGGCGCGGGCCACCACGTGCGCCTGGAGGAGCGCTTCTCCGA
CGACCAGCTGGTGTCCATGTCGGTGCGCGAGCTGAACCGGCAGCTCCGC
GGCTTCAGCAAGGAGGAGGTCATCCGGCTCAAGCAGAAGCGGCGCACGC
TCAAGAACCGCGGCTACGCGCAGTCCTGCCGCTTCAAGCGGGTGCAGCA
GCGGCACATTCTGGAGAGCGAGAAGTGCCAACTCCAGAGCCAGGTGGAG
CAGCTGAAGCTGGAGGTGGGGCGCCTGGCCAAAGAGCGGGACCTGTACA
AGGAGAAATACGAGAAGCTGGCGGGCCGGGGCGGCCCCGGGAGCGCGGG
CGGGGCCGGTTTCCCGCGGGAGCCTTCGCCGCCGCAGGCCGGTCCCGGC
GGGGCCAAGGGCACGGCCGACTTCTTCCTG,

or a nucleic acid sequence that hybridizes to a nucleic acid sequence consisting of SEQ ID NO:6 under stringent hybridization conditions.

In some embodiments, the Tcf3 comprises the amino acid sequence:

(SEQ ID NO: 7)
MNQPQRMAPVGTDKELSDLLDFSMMFPLPVTNGKGRPASLAGAQFGGSG
LEDRPSSGSWGSGDQSSSSFDPSRTFSEGTHFTESHSSLSSSTFLGPGL
GGKSGERGAYASFGRDAGVGGLTQAGFLSGELALNSPGPLSPSGMKGTS
QYYPSYSGSSRRRAADGSLDTQPKKVRKVPPGLPSSVYPPSSGEDYGRD
ATAYPSAKTPSSTYPAPFYVADGSLHPSAELWSPPGQAGFGPMLGGGSS
PLPLPPGSGPVGSSGSSSTFGGLHQHERMGYQLHGAEVNGGLPSASSFS
SAPGATYGGVSSHTPPVSGADSLLGSRGTTAGSSGDALGKALASIYSPD
HSSNNFSSSPSTPVGSPQGLAGTSQWPRAGAPGALSPSYDGGLHGLQSK
IEDHLDEAIHVLRSHAVGTAGDMHTLLPGHGALASGFTSPMSLGGRHAG
LVGGSHPEDGLAGSTSLMHNHAALPSQPGTLPDLSRPPDSYSGLGRAGA
TAAASEIKREEKEDEENTSAADHSEEEKKELKAPRARTSPDEDEDDLLP
PEQKAEREKERRVANNARERLRVRDINEAFKELGRMCQLHLNSEKPQTK
LLILHQAVSVILNLEQQVRERNLNPKAACLKRREEEKVSGVVGDPQMVL
SAPHPGLSEAHNPAGHM,

or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:7.

In some embodiments, the Tcf3 comprises the amino acid sequence:

(SEQ ID NO: 8)
MNQPQRMAPVGTDKELSDLLDFSMMFPLPVTNGKGRPASLAGAQFGGSG
LEDRPSSGSWGSGDQSSSSFDPSRTFSEGTHFTESHSSLSSSTFLGPGL
GGKSGERGAYASFGRDAGVGGLTQAGFLSGELALNSPGPLSPSGMKGTS
QYYPSYSGSSRRRAADGSLDTQPKKVRKVPPGLPSSVYPPSSGEDYGRD
ATAYPSAKTPSSTYPAPFYVADGSLHPSAELWSPPGQAGFGPMLGGGSS
PLPLPPGSGPVGSSGSSSTFGGLHQHERMGYQLHGAEVNGGLPSASSFS
SAPGATYGGVSSHTPPVSGADSLLGSRGTTAGSSGDALGKALASIYSPD
HSSNNFSSSPSTPVGSPQGLAGTSQWPRAGAPGALSPSYDGGLHGLQSK
IEDHLDEAIHVLRSHAVGTAGDMHTLLPGHGALASGFTGPMSLGGRHAG
LVGGSHPEDGLAGSTSLMHNHAALPSQPGTLPDLSRPPDSYSGLGRAGA
TAAASEIKREEKEDEENTSAADHSEEEKKELKAPRARTSSTDEVLSLEE
KDLRDRERRMANNARERVRVRDINEAFRELGRMCQMHLKSDKAQTKLLI
LQQAVQVILGLEQQVRERNLNPKAACLKRREEEKVSGVVGDPQMVLSAP
HPGLSEAHNPAGHM,

or an amino acid sequence that has at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:8.

In some embodiments, the nucleic acid sequence encoding the Tcf3 comprises the nucleic acid sequence:

(SEQ ID NO: 9)
ATGAACCAGCCGCAGAGGATGGCGCCTGTGGGCACAGACAAGGAGCTCA
GTGACCTCCTGGACTTCAGCATGATGTTCCCGCTGCCTGTCACCAACGG
GAAGGGCCGGCCCGCCTCCCTGGCCGGGGCGCAGTTCGGAGGTTCAGGT
CTTGAGGACCGGCCCAGCTCAGGCTCCTGGGGCAGCGGCGACCAGAGCA
GCTCCTCCTTTGACCCCAGCCGGACCTTCAGCGAGGGCACCCACTTCAC
TGAGTCGCACAGCAGCCTCTCTTCATCCACATTCCTGGGACCGGGACTC
GGAGGCAAGAGCGGTGAGCGGGGCGCCTATGCCTCCTTCGGGAGAGACG
CAGGCGTGGGCGGCCTGACTCAGGCTGGCTTCCTGTCAGGCGAGCTGGC
CCTCAACAGCCCCGGGCCCCTGTCCCCTTCGGGCATGAAGGGGACCTCC
CAGTACTACCCCTCCTACTCCGGCAGCTCCCGGCGGAGAGCGGCAGACG
GCAGCCTAGACACGCAGCCCAAGAAGGTCCGGAAGGTCCCGCCGGGTCT
TCCATCCTCGGTGTACCCACCCAGCTCAGGTGAGGACTACGGCAGGGAT
GCCACCGCCTACCCGTCCGCCAAGACCCCCAGCAGCACCTATCCCGCCC
CCTTCTACGTGGCAGATGGCAGCCTGCACCCCTCAGCCGAGCTCTGGAG
TCCCCCGGGCCAGGCGGGCTTCGGGCCCATGCTGGGTGGGGGCTCATCC
CCGCTGCCCCTCCCGCCCGGTAGCGGCCCGGTGGGCAGCAGTGGAAGCA
GCAGCACGTTTGGTGGCCTGCACCAGCACGAGCGTATGGGCTACCAGCT
GCATGGAGCAGAGGTGAACGGTGGGCTCCCATCTGCATCCTCCTTCTCC
TCAGCCCCCGGAGCCACGTACGGCGGCGTCTCCAGCCACACGCCGCCTG
TCAGCGGGGCCGACAGCCTCCTGGGCTCCCGAGGGACCACAGCTGGCAG
CTCCGGGGATGCCCTCGGCAAAGCACTGGCCTCGATCTACTCCCCGGAT
CACTCAAGCAATAACTTCTCGTCCAGCCCTTCTACCCCCGTGGGCTCCC
CCCAGGGCCTGGCAGGAACGTCACAGTGGCCTCGAGCAGGAGCCCCCGG
TGCCTTATCGCCCAGCTACGACGGGGGTCTCCACGGCCTGCAGAGTAAG
ATAGAAGACCACCTGGACGAGGCCATCCACGTGCTCCGCAGCCACGCCG
TGGGCACAGCCGGCGACATGCACACGCTGCTGCCTGGCCACGGGGCGCT
GGCCTCAGGTTTCACCAGTCCCATGTCGCTGGGTGGGCGGCACGCAGGC
CTGGTTGGAGGCAGCCACCCCGAGGACGGCCTCGCAGGCAGCACCAGCC
TCATGCACAACCACGCGGCCCTCCCCAGCCAGCCAGGCACCCTCCCTGA
CCTGTCTCGGCCTCCCGACTCCTACAGTGGGCTAGGGCGAGCAGGTGCC
ACGGCGGCCGCCAGCGAGATCAAGCGGGAGGAGAAGGAGGACGAGGAGA
ACACGTCAGCGGCTGACCACTCGGAGGAGGAGAAGAAGGAGCTGAAGGC
CCCCCGGGCCCGGACCAGCCCAGACGAGGACGAGGACGACCTTCTCCCC
CCAGAGCAGAAGGCCGAGCGGGAGAAGGAGCGCCGGGTGGCCAATAACG
CCCGGGAGCGGCTGCGGGTCCGTGACATCAACGAGGCCTTTAAGGAGCT
GGGGCGCATGTGCCAACTGCACCTCAACAGCGAGAAGCCCCAGACCAAA
CTGCTCATCCTGCACCAGGCTGTCTCGGTCATCCTGAACTTGGAGCAGC
AAGTGCGAGAGCGGAACCTGAATCCCAAAGCAGCCTGTTTGAAACGGCG
AGAAGAGGAAAAGGTGTCAGGTGTGGTTGGAGACCCCCAGATGGTGCTT
TCAGCTCCCCACCCAGGCCTGAGCGAAGCCCACAACCCCGCCGGGCACA
TG,

or a nucleic acid sequence that hybridizes to a nucleic acid sequence consisting of SEQ ID NO:9 under stringent hybridization conditions.

In some embodiments, the nucleic acid sequence encoding the Tcf3 comprises the nucleic acid sequence:

(SEQ ID NO: 10)
ATGAACCAGCCGCAGAGGATGGCGCCTGTGGGCACAGACAAGGAGCTCA
GTGACCTCCTGGACTTCAGCATGATGTTCCCGCTGCCTGTCACCAACGG
GAAGGGCCGGCCCGCCTCCCTGGCCGGGGCGCAGTTCGGAGGTTCAGGT
CTTGAGGACCGGCCCAGCTCAGGCTCCTGGGGCAGCGGCGACCAGAGCA
GCTCCTCCTTTGACCCCAGCCGGACCTTCAGCGAGGGCACCCACTTCAC
TGAGTCGCACAGCAGCCTCTCTTCATCCACATTCCTGGGACCGGGACTC
GGAGGCAAGAGCGGTGAGCGGGGCGCCTATGCCTCCTTCGGGAGAGACG
CAGGCGTGGGCGGCCTGACTCAGGCTGGCTTCCTGTCAGGCGAGCTGGC
CCTCAACAGCCCCGGGCCCCTGTCCCCTTCGGGCATGAAGGGGACCTCC
CAGTACTACCCCTCCTACTCCGGCAGCTCCCGGCGGAGAGCGGCAGACG
GCAGCCTAGACACGCAGCCCAAGAAGGTCCGGAAGGTCCCGCCGGGTCT
TCCATCCTCGGTGTACCCACCCAGCTCAGGTGAGGACTACGGCAGGGAT
GCCACCGCCTACCCGTCCGCCAAGACCCCCAGCAGCACCTATCCCGCCC
CCTTCTACGTGGCAGATGGCAGCCTGCACCCCTCAGCCGAGCTCTGGAG
TCCCCCGGGCCAGGCGGGCTTCGGGCCCATGCTGGGTGGGGGCTCATCC
CCGCTGCCCCTCCCGCCCGGTAGCGGCCCGGTGGGCAGCAGTGGAAGCA
GCAGCACGTTTGGTGGCCTGCACCAGCACGAGCGTATGGGCTACCAGCT
GCATGGAGCAGAGGTGAACGGTGGGCTCCCATCTGCATCCTCCTTCTCC
TCAGCCCCCGGAGCCACGTACGGCGGCGTCTCCAGCCACACGCCGCCTG
TCAGCGGGGCCGACAGCCTCCTGGGCTCCCGAGGGACCACAGCTGGCAG
CTCCGGGGATGCCCTCGGCAAAGCACTGGCCTCGATCTACTCCCCGGAT
CACTCAAGCAATAACTTCTCGTCCAGCCCTTCTACCCCCGTGGGCTCCC
CCCAGGGCCTGGCAGGAACGTCACAGTGGCCTCGAGCAGGAGCCCCCGG
TGCCTTATCGCCCAGCTACGACGGGGGTCTCCACGGCCTGCAGAGTAAG
ATAGAAGACCACCTGGACGAGGCCATCCACGTGCTCCGCAGCCACGCCG
TGGGCACAGCCGGTGACATGCACACGCTGCTGCCTGGCCACGGGGCGCT
GGCCTCAGGTTTCACCGGCCCCATGTCACTGGGCGGGCGGCACGCAGGC
CTGGTTGGAGGCAGCCACCCCGAGGACGGCCTCGCAGGCAGCACCAGCC
TCATGCACAACCACGCGGCCCTCCCCAGCCAGCCAGGCACCCTCCCTGA
CCTGTCTCGGCCTCCCGACTCCTACAGTGGGCTAGGGCGAGCAGGTGCC
ACGGCGGCCGCCAGCGAGATCAAGCGGGAGGAGAAGGAGGACGAGGAGA
ACACGTCAGCGGCTGACCACTCGGAGGAGGAGAAGAAGGAGCTGAAGGC
CCCCCGGGCCCGGACCAGCAGTACGGACGAGGTGCTGTCCCTGGAGGAG
AAAGACCTGAGGGACCGGGAGAGGCGCATGGCCAATAACGCGCGGGAGC
GGGTGCGCGTGCGGGATATTAACGAGGCCTTCCGGGAGCTGGGGCGCAT
GTGCCAGATGCACCTCAAGTCGGACAAAGCGCAGACCAAGCTGCTCATC
CTGCAGCAGGCCGTGCAGGTCATCCTGGGGCTGGAGCAGCAGGTGCGAG
AGCGGAACCTGAATCCCAAAGCAGCCTGTTTGAAACGGCGAGAAGAGGA
AAAGGTGTCAGGTGTGGTTGGAGACCCCCAGATGGTGCTTTCAGCTCCC
CACCCAGGCCTGAGCGAAGCCCACAACCCCGCCGGGCACATG,

or a nucleic acid sequence that hybridizes to a nucleic acid sequence consisting of SEQ ID NO:10 under stringent hybridization conditions.

In order to express a polypeptide or functional nucleic acid, the nucleotide coding sequence may be inserted into appropriate expression vector. Therefore, also disclosed is a non-viral vector comprising a polynucleotide comprising nucleic acid sequences encoding 2, 3, or 4 of the proteins selected from the group consisting of Pdx1, Ng3, Mafa, and Tcf3, wherein the nucleic acid sequences are operably linked to an expression control sequence. In some embodiments, the nucleic acid sequences are operably linked to a single expression control sequence. In other embodiments, the nucleic acid sequences are operably linked to two or more separate expression control sequences.

Methods to construct expression vectors containing genetic sequences and appropriate transcriptional and translational control elements are well known in the art. These methods include in vitro recombinant DNA techniques, synthetic techniques, and in vivo genetic recombination. Such techniques are described in Sambrook et al., Molecular Cloning, A Laboratory Manual (Cold Spring Harbor Press, Plainview, N.Y., 1989), and Ausubel et al., Current Protocols in Molecular Biology (John Wiley & Sons, New York, N.Y., 1989).

Expression vectors generally contain regulatory sequences necessary elements for the translation and/or transcription of the inserted coding sequence. For example, the coding sequence is preferably operably linked to a promoter and/or enhancer to help control the expression of the desired gene product.

The “control elements” or “regulatory sequences” are those non-translated regions of the vector—enhancers, promoters, 5′ and 3′ untranslated regions—which interact with host cellular proteins to carry out transcription and translation. Such elements may vary in their strength and specificity.

A “promoter” is generally a sequence or sequences of DNA that function when in a relatively fixed location in regard to the transcription start site. A “promoter” contains core elements required for basic interaction of RNA polymerase and transcription factors and can contain upstream elements and response elements.

“Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers function to increase transcription from nearby promoters. Enhancers, like promoters, also often contain response elements that mediate the regulation of transcription. Enhancers often determine the regulation of expression.

An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

Promoters used in biotechnology are of different types according to the intended type of control of gene expression. They can be generally divided into constitutive promoters, tissue-specific or development-stage-specific promoters, inducible promoters, and synthetic promoters.

Constitutive promoters direct expression in virtually all tissues and are largely, if not entirely, independent of environmental and developmental factors. As their expression is normally not conditioned by endogenous factors, constitutive promoters are usually active across species and even across kingdoms. Examples of constitutive promoters include CMV, EF1a, SV40, PGK1, Ubc, Human beta actin, and CAG.

Tissue-specific or development-stage-specific promoters direct the expression of a gene in specific tissue(s) or at certain stages of development. For plants, promoter elements that are expressed or affect the expression of genes in the vascular system, photosynthetic tissues, tubers, roots and other vegetative organs, or seeds and other reproductive organs can be found in heterologous systems (e.g. distantly related species or even other kingdoms) but the most specificity is generally achieved with homologous promoters (i.e. from the same species, genus or family). This is probably because the coordinate expression of transcription factors is necessary for regulation of the promoter's activity.

The performance of inducible promoters is not conditioned to endogenous factors but to environmental conditions and external stimuli that can be artificially controlled. Within this group, there are promoters modulated by abiotic factors such as light, oxygen levels, heat, cold and wounding. Since some of these factors are difficult to control outside an experimental setting, promoters that respond to chemical compounds, not found naturally in the organism of interest, are of particular interest. Along those lines, promoters that respond to antibiotics, copper, alcohol, steroids, and herbicides, among other compounds, have been adapted and refined to allow the induction of gene activity at will and independently of other biotic or abiotic factors.

The two most commonly used inducible expression systems for research of eukaryote cell biology are named Tet-Off and Tet-On. The Tet-Off system makes use of the tetracycline transactivator (tTA) protein, which is created by fusing one protein, TetR (tetracycline repressor), found in Escherichia coli bacteria, with the activation domain of another protein, VP16, found in the Herpes Simplex Virus. The resulting tTA protein is able to bind to DNA at specific TetO operator sequences. In most Tet-Off systems, several repeats of such TetO sequences are placed upstream of a minimal promoter such as the CMV promoter. The entirety of several TetO sequences with a minimal promoter is called a tetracycline response element (TRE), because it responds to binding of the tetracycline transactivator protein tTA by increased expression of the gene or genes downstream of its promoter. In a Tet-Off system, expression of TRE-controlled genes can be repressed by tetracycline and its derivatives. They bind tTA and render it incapable of binding to TRE sequences, thereby preventing transactivation of TRE-controlled genes. A Tet-On system works similarly, but in the opposite fashion. While in a Tet-Off system, tTA is capable of binding the operator only if not bound to tetracycline or one of its derivatives, such as doxycycline, in a Tet-On system, the rtTA protein is capable of binding the operator only if bound by a tetracycline. Thus the introduction of doxycycline to the system initiates the transcription of the genetic product. The Tet-On system is sometimes preferred over Tet-Off for its faster responsiveness.

In some embodiments, the nucleic acid sequences encoding Pdx1, Ng3, Mafa, and Tcf3 are operably linked to the same expression control sequence. Alternatively, internal ribosome entry sites (IRES) elements can be used to create multigene, or polycistronic, messages. IRES elements are able to bypass the ribosome scanning model of 5′ methylated Cap dependent translation and begin translation at internal sites. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Disclosed are non-viral vectors containing one or more polynucleotides disclosed herein operably linked to an expression control sequence. Examples of such non-viral vectors include the oligonucleotide alone or in combination with a suitable protein, polysaccharide or lipid formulation. Non-viral methods present certain advantages over viral methods, with simple large scale production and low host immunogenicity being just two. Previously, low levels of transfection and expression of the gene held non-viral methods at a disadvantage; however, recent advances in vector technology have yielded molecules and techniques with transfection efficiencies similar to those of viruses.

Examples of suitable non-viral vectors include, but are not limited to pIRES-hrGFP-2a, pCMV6, pMAX, pCAG, pAd-IRES-GFP, and pCDNA3.0.

The compositions disclosed can be used therapeutically in combination with a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The herein disclosed compositions, including pharmaceutical composition, may be administered in a number of ways depending on whether local or systemic treatment is desired, and on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant.

Methods

Also disclosed are methods of reprogramming skin cells into insulin-producing cells that involve delivering intracellularly into the skin cells a polynucleotide comprising nucleic acid sequences encoding Pdx1, Ng3, Mafa, and Tcf3. In some embodiments, the nucleic acid sequences are present in non-viral vectors. In some embodiments, the nucleic acid sequences are operably linked to an expression control sequence. In other embodiments the nucleic acids are operably linked to two or more expression control sequences.

A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion.

In some embodiments, after transfecting target cells with PMN-T factors, the cells can then pack the transfected genes (e.g. cDNA) into EVs, which can then induce other skin cells to form insulin-producing cells. Therefore, also disclosed is a method of reprogramming skin cells into insulin-producing cells that involves exposing the somatic cell with an extracellular vesicle produced from a cell containing or Pdx1, Ng3, Mafa, and Tcf3.

Therefore, disclosed are methods of reprogramming skin cells into insulin-producing cells that involve exposing the skin cells to extracellular vesicles (EVs) isolated from cells expressing or containing exogenous polynucleotides comprising one or more nucleic acid sequences encoding Pdx1, Ng3, Mafa, and Tcf3. EVs secreted by the donor cells can then collected from the culture medium. These EVs can then be administered to the skin cells to reprogram them into insulin-producing cells. In some embodiments, the donor cells can be any cell from the subject able to produce EVs, including (but not limited to) skin cells (e.g., fibroblasts, keratinocytes, skin stem cells), adipocytes, dendritic cells, peripheral blood mononuclear cells (PBMC), pancreatic cells (e.g., ductal epithelial cells), liver cells (e.g., hepatocytes), immune cells (e.g., T cells, macrophages, myeloid derived suppressor cells).

Exosomes and microvesicles are EVs that differ based on their process of biogenesis and biophysical properties, including size and surface protein markers. Exosomes are homogenous small particles ranging from 40 to 150 nm in size and they are normally derived from the endocytic recycling pathway. In endocytosis, endocytic vesicles form at the plasma membrane and fuse to form early endosomes. These mature and become late endosomes where intraluminal vesicles bud off into an intra-vesicular lumen. Instead of fusing with the lysosome, these multivesicular bodies directly fuse with the plasma membrane and release exosomes into the extracellular space. Exosome biogenesis, protein cargo sorting, and release involve the endosomal sorting complex required for transport (ESCRT complex) and other associated proteins such as Alix and Tsg101. In contrast, microvesicles, are produced directly through the outward budding and fission of membrane vesicles from the plasma membrane, and hence, their surface markers are largely dependent on the composition of the membrane of origin. Further, they tend to constitute a larger and more heterogeneous population of extracellular vesicles, ranging from 150 to 1000 nm in diameter. However, both types of vesicles have been shown to deliver functional mRNA, miRNA and proteins to recipient cells.

In some embodiments, the polynucleotides are delivered to the somatic cells, or the donor cells for EVs, intracellularly via a gene gun, a microparticle or nanoparticle suitable for such delivery, transfection by electroporation, three-dimensional nanochannel electroporation, a tissue nanotransfection device, a liposome suitable for such delivery, or a deep-topical tissue nanoelectroinjection device. In some embodiments, a viral vector can be used. However, in other embodiments, the polynucleotides are not delivered virally.

Electroporation is a technique in which an electrical field is applied to cells in order to increase permeability of the cell membrane, allowing cargo (e.g., reprogramming factors) to be introduced into cells. Electroporation is a common technique for introducing foreign DNA into cells.

Tissue nanotransfection allows for direct cytosolic delivery of cargo (e.g., reprogramming factors) into cells by applying a highly intense and focused electric field through arrayed nanochannels, which benignly nanoporates the juxtaposing tissue cell members, and electrophoretically drives cargo into the cells.

In one embodiment, the disclosed compositions are administered in a dose equivalent to parenteral administration of about 0.1 ng to about 100 g per kg of body weight, about 10 ng to about 50 g per kg of body weight, about 100 ng to about 1 g per kg of body weight, from about 1 μg to about 100 mg per kg of body weight, from about 1 μg to about 50 mg per kg of body weight, from about 1 mg to about 500 mg per kg of body weight; and from about 1 mg to about 50 mg per kg of body weight. Alternatively, the amount of the disclosed compositions administered to achieve a therapeutic effective dose is about 0.1 ng, 1 ng, 10 ng, 100 ng, 1 μg, 10 μg, 100 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 30 mg, 40 mg, 50 mg, 60 mg, 70 mg, 80 mg, 90 mg, 100 mg, 500 mg per kg of body weight or greater.

The disclosed methods can be used to treat various forms of diabetes. In some embodiments, the disclosed compositions and methods are used to treat insulin-dependent (type-1) or insulin-resistant (type-2) diabetes in a subject. For example, the subject can have autoimmune diabetes, pacreatectomy-associated diabetes, or metabolic syndrome conducive to insulin resistance. In some embodiments, the disclosed compositions and methods are used to treat gestational diabetes. In some cases, the method can be used to treat a subject with pre-diabetes. The disclosed methods can be used to treat diseases, disorders, or conditions affected by insulin deficiency, such as pancreatitis or pancreatectomy.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Glycemic Control in Mice with Type 1 Diabetes Induced by Streptozotocin Injection PBS Baseline Comparison

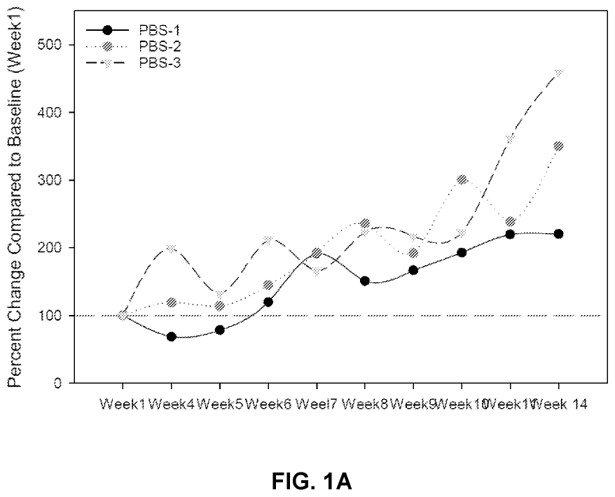

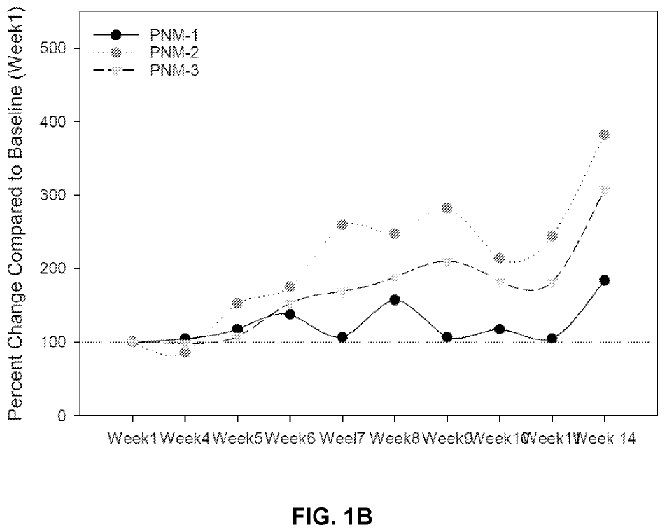

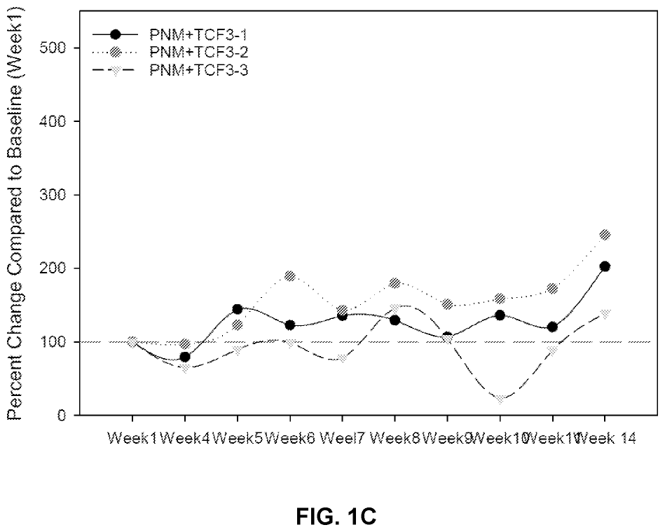

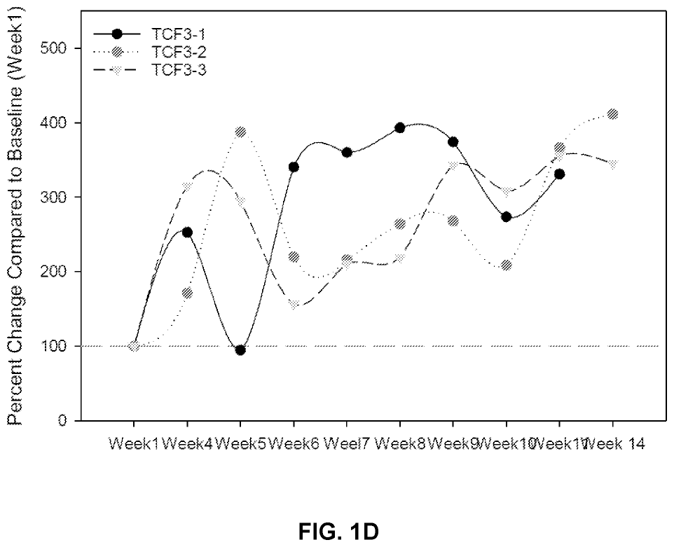

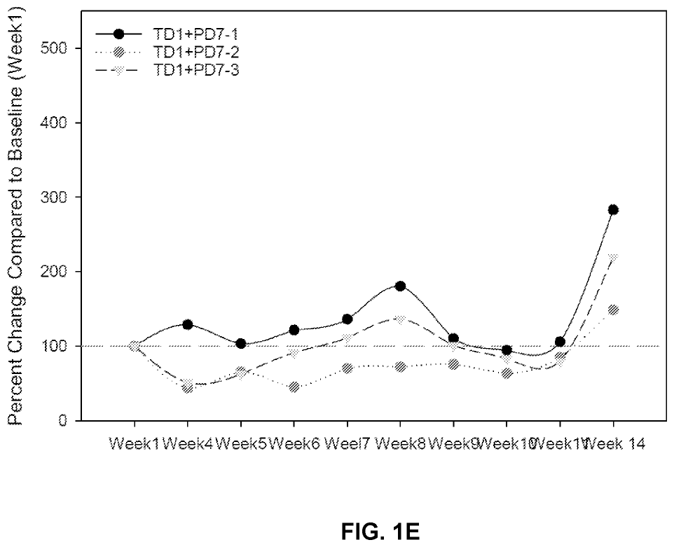

FIGS. 1A to 1E show the results of a study in which diabetic mice were treated once with different genetic cocktails (by deep-topical nanoelectroinjection into the skin). Blood glucose (y-axis) was measured from week 1 (post-treatment) until week 14 (x-axis). The results show that the PNM-T cocktail, either delivered simultaneously or sequentially, was able to support a more controlled glucose level (i.e., more similar to the baseline the mice started with) compared to control/untreated mice and other permutations of the cocktail.

Although the PNM genes (Pdx1, Ngn3, Mafa) have been previously reported to modulate the reprogramming of pancreatic acinar into beta-like cells, no method has been developed for successful non-viral reprogramming of skin tissue into insulin-producing tissue in vivo. The introduction of Tcf3, a transcription factor that plays a fundamental role in modulating skin plasticity, has enabled the development of a skin-tailored reprogramming gene cocktail that can be delivered into the skin and facilitate the establishment of systemic euglycemia in otherwise diabetic/hyperglycemic organisms.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A non-viral vector comprising a first nucleic acid sequences encoding Pdx1, a second nucleic acid sequences encoding Ng3, a third nucleic acid sequences encoding Mafa, and a fourth nucleic acid sequences encoding Tcf3.

2. A non-viral vector of claim 1, wherein each of the first, second, third, and fourth nucleic acid sequence are operably linked to an expression control sequence.

3. The non-viral vector of claim 2, wherein each of the first, second, third, and fourth nucleic acid sequences are operably linked to a single expression control sequence.

4. The non-viral vector of claim 2, wherein the non-viral vector comprises a plasmid selected from the group pIRES-hrGFP-2a, pCMV6, pMAX, pCAG, pAd-IRES-GFP, and pCDNA3.0.

5. The non-viral vector of claim 2, wherein the polynucleotide is encapsulated in a liposome, microparticle or nanoparticle suitable for intracellular delivery.

6. A method of reprogramming a skin cell into an insulin producing cell, comprising
(a) delivering intracellularly into the skin cell Pdx1, Ng3, Mafa, and Tcf3 proteins, or polynucleotides encoding Pdx1, Ng3, Mafa, and Tcf3 or
(b) exposing the skin cell to an extracellular vesicle produced from a cell containing or expressing Pdx1, Ng3, Mafa, and Tcf3 proteins, or polynucleotides encoding Pdx1, Ng3, Mafa, and Tcf3.

7. The method of claim 6, wherein the proteins or polynucleotides are administered sequentially.

8. The method of claim 6, comprising delivering intracellularly into the somatic cell the non-viral vector.

9. The method of claim 6, wherein intracellular delivery comprises three-dimensional nanochannel electroporation.

10. The method of claim 6, wherein intracellular delivery comprises delivery by a tissue nanotransfection device.

11. The method of claim 6, wherein intracellular delivery comprises delivery by a deep-topical tissue nanoelectroinjection device.

12. A method for treating diabetes in a subject, comprising reprogramming an effective amount of skin cells in the subject into an insulin producing cell using the method of claim 6.

13. The method of claim 12, wherein the subject has insulin-dependent diabetes.

14. The method of claim 12, wherein the subject has insulin-resistant diabetes.

15. The method of claim 13, wherein the subject has a fasting blood glucose level between 70 and 130 mg/dL during treatment.


SEQUENCE LISTING (10 sequences)

SEQ ID NO: 1 - protein, 283 aa, Homo sapiens
MNGEEQYYAATQLYKDPCAFQRGPAPEFSASPPACLYMGRQPPPPPPHPFPGALGALEQGSPPDISPYEVPPLADDPAVAHLHHHLPAQLALPHPPAGPFPEGAEPGVLEEPNRVQLPFPWMKSTKAHAWKGQWAGGAYAAEPEENKRTRTAYTRAQLLELEKEFLFNKYISRPRRVELAVMLNLTERHIKIWFQNRRMKWKKEEDKKRGGGTAVGGGGVAEPEQDCAVTSGEELLALPPPPPPGGAVPPAAPVAAREGRLPPGLSASPQPSSVAPRRPQEPR

SEQ ID NO: 2 - DNA, 849 bp, Homo sapiens
atgaacggcgaggagcagtactacgcggccacgcagctttacaaggacccatgcgcgttccagcgaggcccggcgccggagttcagcgccagcccccctgcgtgcctgtacatgggccgccagcccccgccgccgccgccgcacccgttccctggcgccctgggcgcgctggagcagggcagcccccctgacatctccccgtacgaggtgccccccctcgccgacgaccccgcggtggcgcaccttcaccaccacctcccggctcagctcgcgctcccccacccgcccgccgggcccttcccggagggagccgaaccgggcgtcctggaggagcccaaccgcgtccagctgcctttcccatggatgaagtctaccaaagctcacgcgtggaaaggccagtgggcaggcggcgcctacgctgcggagccggaggagaacaagcggacgcgcacggcctacacgcgcgcacagctgctagagctggagaaggagttcctattcaacaagtacatctcacggccgcgccgggtggagctggctgtcatgttgaacttgaccgagagacacatcaagatctggttccaaaaccgccgcatgaagtggaaaaaggaggaggacaagaagcgcggcggcgggacagctgtcgggggtggcggggtcgcggagcctgagcaggactgcgccgtgacctccggcgaggagcttctggcgctgccgccgccgccgccccccggaggtgctgtgccgcccgctgcccccgttgccgcccgagagggccgcctgccgcctggccttagcgcgtcgccacagccctccagcgtcgcgcctcggcggccgcaggaaccacga

SEQ ID NO: 3 - protein, 214 aa, Homo sapiens [sequence omitted]

SEQ ID NO: 4 - DNA, 642 bp, Homo sapiens
atgacgcctcaaccctcgggtgcgcccactgtccaagtgacccgtgagacggagcggtccttccccagagcctcggaagacgaagtgacctgccccacgtccgccccgcccagccccactcgcacacgggggaactgcgcagaggcggaagagggaggctgccgaggggccccgaggaagctccgggcacggcgcgggggacgcagccggcctaagagcgagttggcactgagcaagcagcgacggagtcggcgaaagaaggccaacgaccgcgagcgcaatcgaatgcacaacctcaactcggcactggacgccctgcgcggtgtcctgcccaccttcccagacgacgcgaagctcaccaagatcgagacgctgcgcttcgcccacaactacatctgggcgctgactcaaacgctgcgcatagcggaccacagcttgtacgcgctggagccgccggcgccgcactgcggggagctgggcagcccaggcggttcccccggggactgggggtccctctactccccagtctcccaggctggcagcctgagtcccgccgcgtcgctggaggagcgacccgggctgctgggggccacctcttccgcctgcttgagcccaggcagtctggctttctcagattttctg

SEQ ID NO: 5 - protein, 353 aa, Homo sapiens
MAAELAMGAELPSSPLAIEYVNDFDLMKFEVKKEPPEAERFCHRLPPGSLSSTPLSTPCSSVPSSPSFCAPSPGTGGGGGAGGGGGSSQAGGAPGPPSGGPGAVGGTSGKPALEDLYWMSGYQHHLNPEALNLTPEDAVEALIGSGHHGAHHGAHHPAAAAAYEAFRGPGFAGGGGADDMGAGHHHGAHHAAHHHHAAHHHHHHHHHHGGAGHGGGAGHHVRLEERFSDDQLVSMSVRELNRQLRGFSKEEVIRLKQKRRTLKNRGYAQSCRFKRVQQRHILESEKCQLQSQVEQLKLEVGRLAKERDLYKEKYEKLAGRGGPGSAGGAGFPREPSPPQAGPGGAKGTADFFL

SEQ ID NO: 6 - DNA, 1059 bp, Homo sapiens
atggccgcggagctggcgatgggcgccgagctgcccagcagcccgctggccatcgagtacgtcaacgacttcgacctgatgaagttcgaggtgaagaaggagcctcccgaggccgagcgcttctgccaccgcctgccgccaggctcgctgtcctcgacgccgctcagcacgccctgctcctccgtgccctcctcgcccagcttctgcgcgcccagcccgggcaccggcggcggcggcggcgcggggggcggcggcggctcgtctcaggccgggggcgcccccgggccgccgagcgggggccccggcgccgtcgggggcacctcggggaagccggcgctggaggatctgtactggatgagcggctaccagcatcacctcaaccccgaggcgctcaacctgacgcccgaggacgcggtggaggcgctcatcggcagcggccaccacggcgcgcaccacggcgcgcaccacccggcggccgccgcagcctacgaggctttccgcggcccgggcttcgcgggcggcggcggagcggacgacatgggcgccggccaccaccacggcgcgcaccacgccgcccaccaccaccacgccgcccaccaccaccaccaccaccaccaccaccatggcggcgcgggacacggcggtggcgcgggccaccacgtgcgcctggaggagcgcttctccgacgaccagctggtgtccatgtcggtgcgcgagctgaaccggcagctccgcggcttcagcaaggaggaggtcatccggctcaagcagaagcggcgcacgctcaagaaccgcggctacgcgcagtcctgccgcttcaagcgggtgcagcagcggcacattctggagagcgagaagtgccaactccagagccaggtggagcagctgaagctggaggtggggcgcctggccaaagagcgggacctgtacaaggagaaatacgagaagctggcgggccggggcggccccgggagcgcgggcggggccggtttcccgcgggagccttcgccgccgcaggccggtcccggcggggccaagggcacggccgacttcttcctg

SEQ ID NO: 7 - protein, 654 aa, Homo sapiens [sequence omitted]

SEQ ID NO: 8 - protein, 651 aa, Homo sapiens
MNQPQRMAPVGTDKELSDLLDFSMMFPLPVTNGKGRPASLAGAQFGGSGLEDRPSSGSWGSGDQSSSSFDPSRTFSEGTHFTESHSSLSSSTFLGPGLGGKSGERGAYASFGRDAGVGGLTQAGFLSGELALNSPGPLSPSGMKGTSQYYPSYSGSSRRRAADGSLDTQPKKVRKVPPGLPSSVYPPSSGEDYGRDATAYPSAKTPSSTYPAPFYVADGSLHPSAELWSPPGQAGFGPMLGGGSSPLPLPPGSGPVGSSGSSSTFGGLHQHERMGYQLHGAEVNGGLPSASSFSSAPGATYGGVSSHTPPVSGADSLLGSRGTTAGSSGDALGKALASIYSPDHSSNNFSSSPSTPVGSPQGLAGTSQWPRAGAPGALSPSYDGGLHGLQSKIEDHLDEAIHVLRSHAVGTAGDMHTLLPGHGALASGFTGPMSLGGRHAGLVGGSHPEDGLAGSTSLMHNHAALPSQPGTLPDLSRPPDSYSGLGRAGATAAASEIKREEKEDEENTSAADHSEEEKKELKAPRARTSSTDEVLSLEEKDLRDRERRMANNARERVRVRDINEAFRELGRMCQMHLKSDKAQTKLLILQQAVQVILGLEQQVRERNLNPKAACLKRREEEKVSGVVGDPQMVLSAPHPGLSEAHNPAGHM

SEQ ID NO: 9 - DNA, 1962 bp, Homo sapiens
atgaaccagccgcagaggatggcgcctgtgggcacagacaaggagctcagtgacctcctggacttcagcatgatgttcccgctgcctgtcaccaacgggaagggccggcccgcctccctggccggggcgcagttcggaggttcaggtcttgaggaccggcccagctcaggctcctggggcagcggcgaccagagcagctcctcctttgaccccagccggaccttcagcgagggcacccacttcactgagtcgcacagcagcctctcttcatccacattcctgggaccgggactcggaggcaagagcggtgagcggggcgcctatgcctccttcgggagagacgcaggcgtgggcggcctgactcaggctggcttcctgtcaggcgagctggccctcaacagccccgggcccctgtccccttcgggcatgaaggggacctcccagtactacccctcctactccggcagctcccggcggagagcggcagacggcagcctagacacgcagcccaagaaggtccggaaggtcccgccgggtcttccatcctcggtgtacccacccagctcaggtgaggactacggcagggatgccaccgcctacccgtccgccaagacccccagcagcacctatcccgcccccttctacgtggcagatggcagcctgcacccctcagccgagctctggagtcccccgggccaggcgggcttcgggcccatgctgggtgggggctcatccccgctgcccctcccgcccggtagcggcccggtgggcagcagtggaagcagcagcacgtttggtggcctgcaccagcacgagcgtatgggctaccagctgcatggagcagaggtgaacggtgggctcccatctgcatcctccttctcctcagcccccggagccacgtacggcggcgtctccagccacacgccgcctgtcagcggggccgacagcctcctgggctcccgagggaccacagctggcagctccggggatgccctcggcaaagcactggcctcgatctactccccggatcactcaagcaataacttctcgtccagcccttctacccccgtgggctccccccagggcctggcaggaacgtcacagtggcctcgagcaggagcccccggtgccttatcgcccagctacgacgggggtctccacggcctgcagagtaagatagaagaccacctggacgaggccatccacgtgctccgcagccacgccgtgggcacagccggcgacatgcacacgctgctgcctggccacggggcgctggcctcaggtttcaccagtcccatgtcgctgggtgggcggcacgcaggcctggttggaggcagccaccccgaggacggcctcgcaggcagcaccagcctcatgcacaaccacgcggccctccccagccagccaggcaccctccctgacctgtctcggcctcccgactcctacagtgggctagggcgagcaggtgccacggcggccgccagcgagatcaagcgggaggagaaggaggacgaggagaacacgtcagcggctgaccactcggaggaggagaagaaggagctgaaggccccccgggcccggaccagcccagacgaggacgaggacgaccttctccccccagagcagaaggccgagcgggagaaggagcgccgggtggccaataacgcccgggagcggctgcgggtccgtgacatcaacgaggcctttaaggagctggggcgcatgtgccaactgcacctcaacagcgagaagccccagaccaaactgctcatcctgcaccaggctgtctcggtcatcctgaacttggagcagcaagtgcgagagcggaacctgaatcccaaagcagcctgtttgaaacggcgagaagaggaaaaggtgtcaggtgtggttggagacccccagatggtgctttcagctccccacccaggcctgagcgaagcccacaaccccgccgggcacatg

SEQ ID NO: 10 - DNA, 1953 bp, Homo sapiens
atgaaccagccgcagaggatggcgcctgtgggcacagacaaggagctcagtgacctcctggacttcagcatgatgttcccgctgcctgtcaccaacgggaagggccggcccgcctccctggccggggcgcagttcggaggttcaggtcttgaggaccggcccagctcaggctcctggggcagcggcgaccagagcagctcctcctttgaccccagccggaccttcagcgagggcacccacttcactgagtcgcacagcagcctctcttcatccacattcctgggaccgggactcggaggcaagagcggtgagcggggcgcctatgcctccttcgggagagacgcaggcgtgggcggcctgactcaggctggcttcctgtcaggcgagctggccctcaacagccccgggcccctgtccccttcgggcatgaaggggacctcccagtactacccctcctactccggcagctcccggcggagagcggcagacggcagcctagacacgcagcccaagaaggtccggaaggtcccgccgggtcttccatcctcggtgtacccacccagctcaggtgaggactacggcagggatgccaccgcctacccgtccgccaagacccccagcagcacctatcccgcccccttctacgtggcagatggcagcctgcacccctcagccgagctctggagtcccccgggccaggcgggcttcgggcccatgctgggtgggggctcatccccgctgcccctcccgcccggtagcggcccggtgggcagcagtggaagcagcagcacgtttggtggcctgcaccagcacgagcgtatgggctaccagctgcatggagcagaggtgaacggtgggctcccatctgcatcctccttctcctcagcccccggagccacgtacggcggcgtctccagccacacgccgcctgtcagcggggccgacagcctcctgggctcccgagggaccacagctggcagctccggggatgccctcggcaaagcactggcctcgatctactccccggatcactcaagcaataacttctcgtccagcccttctacccccgtgggctccccccagggcctggcaggaacgtcacagtggcctcgagcaggagcccccggtgccttatcgcccagctacgacgggggtctccacggcctgcagagtaagatagaagaccacctggacgaggccatccacgtgctccgcagccacgccgtgggcacagccggtgacatgcacacgctgctgcctggccacggggcgctggcctcaggtttcaccggccccatgtcactgggcgggcggcacgcaggcctggttggaggcagccaccccgaggacggcctcgcaggcagcaccagcctcatgcacaaccacgcggccctccccagccagccaggcaccctccctgacctgtctcggcctcccgactcctacagtgggctagggcgagcaggtgccacggcggccgccagcgagatcaagcgggaggagaaggaggacgaggagaacacgtcagcggctgaccactcggaggaggagaagaaggagctgaaggccccccgggcccggaccagcagtacggacgaggtgctgtccctggaggagaaagacctgagggaccgggagaggcgcatggccaataacgcgcgggagcgggtgcgcgtgcgggatattaacgaggccttccgggagctggggcgcatgtgccagatgcacctcaagtcggacaaagcgcagaccaagctgctcatcctgcagcaggccgtgcaggtcatcctggggctggagcagcaggtgcgagagcggaacctgaatcccaaagcagcctgtttgaaacggcgagaagaggaaaaggtgtcaggtgtggttggagacccccagatggtgctttcagctccccacccaggcctgagcgaagcccacaaccccgccgggcacatg